(19) United States
(12) Patent Application Publication
(10) Pub. No.: US 2016/0287575 A1
(43) Pub. Date: Oct. 6, 2016

(54) COMPOSITIONS AND METHODS FOR THE TREATMENT OF DISEASES INVOLVING HIPPO PATHWAY

(71) Applicant: DUKE UNIVERSITY, Durham, NC (US)

(72) Inventors: Corinne M. Linardic, Durham, NC (US); Lisa E.S. Crose, Durham, NC (US); Jen-Tsan Chi, Chapel Hill, NC (US)

(73) Assignee: Duke University, Durham, NC (US)

(21) Appl. No.: 15/036,923

(22) PCT Filed: Nov. 14, 2014
(86) PCT No.: PCT/US2014/065689

§ 371 Date: May 16, 2016

Related U.S. Application Data
(60) Provisional application No. 61/904,060, filed on Nov. 14, 2013.

(51) Int. Cl. A61K 31/475; A61N 5/10; G01N 33/68; G01N 33/574; A61K 45/06; A61K 31/409

(52) U.S. Cl. CPC A61K 31/475; A61K 45/06; A61K 31/409; G01N 33/6893; G01N 33/57496; A61N 5/10; G01N 2333/47; G01N 2333/96433

(57) ABSTRACT

Provided herein are interfering molecules and pharmaceutical combinations comprising such interfering molecules that modulate the Hippo signaling pathway. Also provided are methods of treating and preventing a disease or condition associated disruption of a Hippo signaling pathway component, such as skeletal muscle disorders and cancers, where the method comprises administering to a subject in need thereof an interfering molecule or pharmaceutical composition described herein.

### CROSS-REFERENCE TO RELATED APPLICATIONS

This application claims the benefit of U.S. Provisional Application No. 61/904,060, filed Nov. 14, 2013, which is incorporated by reference as if set forth in its entirety.

### STATEMENT REGARDING FEDERALLY SPONSORED RESEARCH OR DEVELOPMENT

This invention was made with government support under R01CA122706 and R01CA125618, awarded by the National Institutes of Health. The government has certain rights in the invention.

### BACKGROUND

Rhabdomyosarcomas (RMS) are malignancies of the skeletal muscle lineage that are the most common soft tissue sarcomas of childhood and adolescence. While there are several histologic variants, including embryonal (eRMS) and alveolar RMS (aRMS), aRMS is the most difficult to cure, with a 5-year survival of <50%. Many aRMS tumors bear a signature chromosomal trans location resulting in the expression of a PAX3-FOXO1 fusion gene, which reactivates pro-proliferative embryonic skeletal muscle signaling pathways. PAX-FOXO1-positive RMS shows a higher propensity to metastasize and reduced survival after currently available treatments when compared with fusion-negative tumors. Children having metastatic PAX3-FOXO1-positive aRMS have the worst outcome, with a 5-year survival of <10%. Few therapeutic options are available for high risk patients having PAX3-FOXO1-positive aRMS. Understanding of the mechanisms by which the fusion product impacts malignancy and how to interfere with these mechanisms or the fusion products themselves remains insufficient.

### SUMMARY OF THE INVENTION

In a first aspect, provided herein is a method of treating a disease in a subject in need thereof comprising administering to said subject a therapeutically effective amount of at least one compound capable of modulating at least one Hippo signaling pathway component, whereby administration of the at least one compound treats a disease or condition associated with a disrupted Hippo signaling pathway. The disease can be selected from the group consisting of a cancer, skeletal muscle disorder, myopathy, muscular dystrophy, myotonic dystrophy, and chronic obstructive pulmonary disorder. The disease can be muscular dystrophy. The muscular dystrophy can be selected from the group consisting of Becker muscular dystrophy, Duchenne muscular dystrophy, Emery-Dreifuss muscular dystrophy, Facioscapulohumoeral muscular dystrophy, Myotonia congentia, and myotonic dystrophy. Duchenne muscular dystrophy. In some cases, the muscular dystrophy is Duchenne muscular dystrophy. Treating can he selected from the group consisting of ameliorating a disease or symptom of a disease, slowing the progression of a disease, and preventing the progression of a disease. In some cases, the cancer selected from the group consisting of fibrosarcoma, rhabdomyosarcoma, myxosarcoma, liosarcoma, chondrosarcoma, osteogenic sarcoma or chordosarcoma, angiosarcoma, Ewing's sarcoma, endotheliosardcoma, lympangiosarcoma, synoviosarcoma, and mesothelisosarcoma. The subject can be human. The at least one compound can be a porphyrin selected from the group consisting of verteporfin, protoporphyrin IX, and hematoporphyrin, or a derivative thereof. The at least one compound can be a cross-linked polypeptide. The at least one compound can be an antisense RNA molecule. The at least one compound can modulate a Hippo signaling pathway component selected from the group consisting of RASSF4, MST1, PAX3-FOXO1, LATS1, LATS2, YAP, and TAZ.

In some cases, the method further comprises administering a therapeutically effective amount of a chemotherapeutic agent to the subject. The chemotherapeutic agent can be selected from the group consisting of cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum 5-fluorouracil, vincristin, vinblastin, and methotrexate.

The method can further comprise exposing the subject to radiation. The radiation can be delivered locally to a site of the cancer. The radiation can be whole body radiation. The radiation can he selected from the group consisting of gamma-rays, x-rays, accelerated protons, microwave radiation, UV radiation, and directed delivery of radioisotopes to tumor cells, or combinations thereof.

In another aspect, provided herein is a method of diagnosing a disease in a subject comprising (a) detecting an expression level of a Hippo signaling pathway component in a biological sample of the subject; and (b) comparing the detected expression level to an expression level of the Hippo signaling pathway component detected in a biological sample of a control subject having a known disease or condition associated with a disrupted Hippo signaling pathway. The Hippo signaling pathway component can be selected from the group consisting of PAX3-FOXO1, RASSF4, MST1, LATS1, LATS2, YAP, and TAZ. The Hippo signaling pathway component can be RASSF4. The Hippo signaling pathway component can be MST1. The biological sample can be a tissue sample obtained from a tissue selected from the group consisting of muscle, brain, lung, liver, spleen, kidney, lymph node, small intestine, blood cells, pancreas, colon, stomach, cervix, breast, endometrium, prostate, testicle, ovary, skin, head, neck, esophagus, oral tissue, bone marrow, and blood. Detecting can comprise measuring a nucleic acid or polypeptide level of at least one of PAX3-FOXO1, RASSF4, MST1, LATS1, LATS2, YAP or TAZ. Measuring can comprise a technique selected from the group consisting of polymerase chain reaction, immunohistochemistry, and ELISA. Detecting can comprise identifying a subcellular location of RASSF4.

In some cases, the method further comprises evaluating the stability of a nucleic acid encoding PAX3-FOXO1, RASSF4, MST1, LATS1, LATS2, YAP or TAZ. The disease or condition of the control subject can he selected from the group consisting of cancer, myopathy, muscular dystrophy, myotonic dystrophy, and chronic obstructive pulmonary disorder. The muscular dystrophy can be selected from the group consisting of Becker muscular dystrophy, Duchenne muscular dystrophy, Emery-Dreifuss muscular dystrophy, Facioscapulohumoeral muscular dystrophy, Myotonia congentia, and myotonic dystrophy. In some cases, the muscular dystrophy is Duchenne muscular dystrophy. In some cases, the cancer is selected from the group consisting of rhabdomyoma, fibroma, lipoma, teratoma, Kaposi's sarcoma, fibrosarcoma, rhabdomyosarcoma, myxosarcoma, liosarcoma, chondrosarcoma, osteogenic sarcoma, chordosarcoma, liposarcoma, angiosarcoma, leiomysarcoma, Ewing's sarcoma, endotheliosardcoma, lympangiosarcoma, synoviosarcoma, and mesothelisosarcoma.

### BRIEF DESCRIPTION OF THE DRAWINGS

FIG. 1 is a schematic of the HSMM^{PF+H+M} model of alveolar RMS (aRMS).

FIG. 2 demonstrates upregulation of RASSF4 in PAX3-FOXO1-positive aRMS cells and tumors. (A) Left: Expression profile of HSMM control cells (Vpre) compared with PAX3-FOXO1-expressing HSMM presenescent (PFpre) or postseriescent (PFpost) cells. Right: Semiquantitative RT-PCR validation of select genes identified in the microarray. (B) PAX3-FOXO1-expressing aRMS cells expressed more RASSF4 than eRMS cells or HSMMs, as measured by qPCR and immunoblotting. *P<0.05; **P<0.005. Labels for cell lines correspond to qPCR and immunoblotting. Actin was used as a loading control. (C) HSMM-based model of aRMS displayed enhanced RASSF4 expression in a PAX3-FOXO1-dependent manner as measured by qPCR. *P<0.05; **P<0.005. (D) PAX3-FOXO1-positive primary human aRMS tumors expressed more RASSF4 than fusion-negative aRMS or eRMS. Error bars represent SEM. *P<0.0001; #P=0.0004; Mann-Whitney U test. Median-centered log2 values are shown, and microarray data were obtained from the Oncogenomics database. (E) RMS patient survival based on RASSF4 expression. The median RASSF4 expression value for RMS was the threshold for high versus low RASSF4 expression. High RASSF4, n=73, Low RASSF4, n=73. P value is based on log-rank test analysis. (F) dRASSF mutation genetically suppressed PAX7-FOXO1 pathogenicity in a Drosophila aRMS model. PAX7-FOXO1 expression in differentiating larval muscle causes semilethality, as PAX7-FOXO1 adults comprise only 9% of F1 adults (n=170). (In Mendelian ratios, the F1 population should be composed of 50% wild-type and 50% PAX7-FOXO1 adults). The Df(3RExcel)6193 chromosomal deletion and dRASSFDG30608 and dRASSFA531 loss-of-function alleles suppressed PAX7-FOXO1-induced lethality. Groucho511 (Gro511) is an unrelated mutation included as a representative example of a nonsuppressor. Df(3R)Exel6193, n=72; dRASSFA531, n=130; dRASSFDG30608, n=66; and Gro511, n=116.

FIG. 3 depicts regulation of RASSF4 5′ enhancer by PAX3-FOXO1. (A) A putative PAX3-FOXO1 binding site 5′ to the RASSF4 gene on human chromosome 10. Figure was derived from publically available data (Cao et al., Cancer Res. (2010)). (B) PAX3 and PAX3-FOXO1 directly regulated the RASSF4 5′ enhancer. RD cells were cotransfected with Renilla, a luciferase vector lacking an enhancer, or the RASSF4 5′ enhancer, along with vector, wild-type PAX3, or PAX3-FOXO1. *P<0.001 compared with vector-expressing cells; #P<0.005 compared with PAX3-expressing

FIG. 4 depicts cell proliferation and senescence inhibition in PAX3-FOXO1 aRMS cells. (A) RASSF4 shRNA validation in HSMM^{PF+H+M} cells. Knockdown was measured by immunoblot analysis for endogenous RASSF4 and actin, which was used as a loading control. pLKO.1 was used as a control vector. (B) Senescence induction in PAX3-FOXO1-expressing HSMM cells. Quantitation of β-gal staining of HSMMV or HSMMPF cells transduced with control vector or RASSF4 shRNAs. *P≦0.01. (C) Loss of RASSF4 in HSMM^{PF+H+M} cells caused deficient cell proliferation, as measured by hemocytometric counts over 5 days of growth in culture and (D) BrdU assay. *P<0.0001. (E) RASSF4-deficient HSMM^{PF+H+M} cells displayed cell shape change, elevated β-gal staining (insets, mean±SD; scale bars: 125 μm), cell cycle arrest (F), and p21 upregulation (G). Error bars represent SD in B and D.

FIG. 5 demonstrates that RASSF4 knockdown in aRMS xenografts inhibits tumor growth. (A) RASSF4 knockdown tumors displayed a delay in tumor progression, as measured by their time to maximum tumor burden. (B) qPCR validation of RASSF4 knockdown in aRMS xenografts. Each bar represents an individual xenograft. RASSF4 knockdown in aRMS xenografts led to multinucleated cells and mitotic defects. Tumor cell morphologic changes were examined by H&E staining (C), and multinucleated cells (D, left, arrowheads) were quantified (D, right). Error bars represent SD. *P=0.017. Scale bars: 25 μm.

FIG. 6 demonstrates that RASSF4 regulates MST1 to inhibit the Hippo pathway in aRMS. (A) Domain architecture of HA-RASSF4 constructs. (B) RASSF4 associated with MST1 in aRMS cells. Anti-HA immunoprecipitates from aRMS cells expressing HA-RASSF4 or an empty vector were examined for coprecipitation of MST1 or pan H-, K-, and N-Ras by immunoblotting. (C) RASSF4-MST1 association was dependent on the RASSF4 SARAH domain. Anti-HA immunoprecipitates from HSMM^{PF+H+M} cells expressing HA-RASSF4, HA-RASSF4ΔSARAH, or vector were used to examine the association with MST1 or pan Ras by immunoblotting. These results were confirmed by immunopurifying endogenous MST1 and by blotting for HA-RASSF4, HA-RASSF4ΔSARAH (D, top), or endogenous RASSF4 (E). (F) Exogenous MST1 expression induced aRMS cell senescence, which was partially inhibited by HA-RASSF4 expression. Error bars represent SD. *P<0.0001 compared with vector expressed alone. #P=0.0005 compared with MST1-expressing cells. (C) RASSF4 suppressed MST1 signaling to MOB1. HSMM^{PF+H+M} cells expressing vector or MST1K59R, vector, or RASSF4 shRNA were cultured in the presence or absence of nocodazole. Protein lysates from these cells were analyzed by immunoblotting. (H) MST1 K59R partially blocked G0/G1 accumulation in RASSF4-deficient cells as measured by cell cycle analysis. (I) MST1K59R prevented senescence induction caused by RASSF4 loss as measured by β-gal assay, *P<0.00005 compared with vector control cells; #P<0.005 compared with cells with RASSF4 knockdown alone.

FIG. 7 demonstrates upregulation of YAP in aRMS. (A) Phosphorylated MST1/2 (MST1 T183, MST2 T180), MST1, phosphorylated (S127) YAP, and YAP levels in HSMMs (proliferating and differentiated) and RMS cell lines as measured by immunoblot analysis. ERK1 was used as a loading control. (B) Left: Representative images from RMS TMAs immunostained for YAP protein. Scale bars: 100 μm. Right: Quantitation of YAP-immunostained RMS TMAs. Muscle, n=11; eRMS, n=58; aRMS, n=72. Error bars represent SEM. *P<0.0001, Mann-Whitney test. (C) YAP1 shRNA knockdown validation in HSMM^{PF+H+M} and Rh28 cell lines as measured by immunoblot analysis. YAP1 knockdown aRMS cells were defective in proliferation, as measured by BrdU incorporation (D). YAP1 loss induced senescence in aRMS cells as measured by β-gal staining (E). (F) Model of RASSF4-mediated suppression of the Hippo pathway in aRMS. PAX3-FOXO1 transcriptionally upregulates RASSF4 through a 5′ enhancer region. Subsequent RASSF4 protein associates with MST1 and inhibits signaling to MOB1. While YAP expression is elevated in aRMS cells and tumors, a connection between RASSF4 and YAP remains to be elucidated. Both MST1 inhibition and YAP expression support cell proliferation and senescence evasion to promote tumorigenesis in aRMS cells.

FIG. 8 presents RASSF expression profiles in pediatric tumor cell lines and xenografts. Median-centered Log2 values of pediatric cancer cell lines and xenographs, as obtained from home.ccr.cancer.gov/oncoloy/oncogenomics on the World Wide Web. ALL, acute lymphoblastic leukemia; aRMS, alveolar rhabdomyosarcoma; eRMS, emrbyonal rhabdomyosarcoma; EWS, Ewing's sarcoma; EP, ependymoma; MB, meduloblastoma; NB, neuroblastoma; OS, osteosarcoma; SkM, normal skeletal muscle. No data was available for RASSFs 3, 9, and 10.

FIG. 9 presents data for RASSF4 shRNA validation. (A) Constitutive RASSF4 shRNA validation in primary HSMM cells expressing vector (Vpre) or PAX3-FOXO1 (PFpre), as measured by semi-quantitative PCR. Validation of doxycycline-inducible RASSF4 shRNA in vitro in Rh28 cells by semi-quantitative RT-PCR, p21 immunoblot (B); endogenous RASSF4 levels in doxycycline-treated cells (C); and BrdU incorporation assay (D). (E) Rh28 xenografts in the absence of doxycycline have no effect on RASSF4 expression levels.

FIG. 10 demonstrates that RASSF4-deficient cells display increased STS-induced MST1 and caspase 3 cleavage. (A) RASSF4 deficiency causes MST1 phosphorylation and cleavage upon staurosporine (STS) treatment. Cells expressing FLAG-MST1 K59R were treated for 1 hour with 1 μM STS and examined for phosphor-MST1/2 and total MST1 by immunoblot. (B) Upon 1 hour treatment with 1 μM STS, RASSF4-deficient aRMS cells display higher cleaved caspase 3 as measured by immunoblot. In A and B, actin was used as a loading control.

FIG. 11 demonstrates CTGF levels in aRMS cells and YAP knockdown cells. (A) CTGF levels in RMS cells as measured using real-time PCR. (B) CTGF levels are unchanged in YAP-deficient aRMS cells.

FIG. 12 presents data demonstrating the effects of verteporfin and vincristine treatment on tumor volume and growth rate. (A) Tumor volume at day 21 for negative control (PBS) mice and mice treated with verteporfin (100 mg/kg, 3 times per week), vincristine, or Vincristine (1 mg/kg, 1 time per week), or a combination of Verteporfin and Vincristine (Vincristine 1 mg/kg 1 time per week. Verteporfin 100 mg/kg 2 times per week). (B) Time (days) for tumors to quadruple in size.

FIG. 13 is a graph demonstrating changes in tumor size as a function of time (days) for negative control (PBS) mice and mice treated with verteporfin (100 mg/kg, 3 times per week), vincristine, or Vincristine (1 mg/kg, 1 time per week), or a combination of Verteporfin and Vincristine (Vincristine 1 mg/kg 1 time per week, Verteporfin 100 mg/kg 2 times per week).

### DETAILED DESCRIPTION OF THE INVENTION

All publications, patents, and patent applications mentioned in this specification are herein incorporated by reference in their entirety as if each individual publication, patent, and patent application was specifically and individually indicated to be incorporated by reference.

Hope for future anti-sarcoma therapies and therapies for a variety of skeletal muscle disorders or muscle wasting diseases may rest in large part on interventions that recruit antitumor immune mechanisms, induce myogenic differentiation, or interfere with specific cellular and molecular mechanisms that drive the malignant behavior of sarcoma cells. Without being bound by any theory or mechanism, it is believed that administration of a compound or pharmaceutical composition described herein can slow or halt proliferation of sarcoma cells by down-regulating cell cycle genes and induce myogenic differentiation of sarcoma cells by up-regulating myogenic genes.

Methods of Treating, Preventing and Ameliorating a Disease

Accordingly, in a first aspect of the present disclosure, provided herein are methods of treating, ameliorating, or preventing the progression of a disease or condition in a subject comprising, consisting of, or consisting essentially of administering to the subject a therapeutically effective amount of an interfering molecule, or pharmaceutical compositions thereof, wherein the interfering molecule is capable of modulating the Hippo pathway. In exemplary embodiments, the disease or condition is associated with a disruption in Hippo pathway signal transduction, and the pharmaceutical composition thereof comprises an effective amount of at least one compound or agent effective to modulate, disrupt, interfere with, or otherwise alter Hippo pathway signal transduction, and a pharmaceutically acceptable carrier.

As used herein, the terms “administering” and “administered” refer to administration of an effective amount of an interfering molecule of the present disclosure. As used herein, the terms “therapeutic amount,” “effective amount,” and “therapeutically effective amount” refer to an amount of a molecule, compound, or pharmaceutical composition effective to elicit a beneficial response to the active ingredient present in the composition, thereby preventing onset or progression of a disease, or treating a disease upon administration of the composition to a subject in need thereof. In accordance with good clinical practice, it is preferred to administer the instant compounds at a concentration level which will produce effective beneficial effects without causing any harmful or untoward side effects.

Preferably, administration is oral or parenteral. By “parenteral” is meant intravenous, subcutaneous or intramuscular administration. In the methods of the subject disclosure, the interfering molecules of the present disclosure may be administered alone, simultaneously, or sequentially with one or more other interfering molecule and/or cytotoxic, chemotherapeutic or anti-cancer agents or, in either order. It will be appreciated that the actual preferred method and order of administration will vary according to, inter alia, the particular preparation of interfering molecules being utilized, the particular formulation(s) of the one or more other interfering molecules being utilized. The optimal method and order of administration of the compounds of the disclosure for a given set of conditions can be ascertained by those skilled in the art using conventional techniques and in view of the information set out herein. In addition, the terms “administering” and “administered” refer to oral sublingual, buccal, transnasal, transdermal, rectal, intramascular, intravenous, intraventricular, intrathecal, and subcutaneous routes. Systemic delivery of an interfering molecule/compound may be necessary for cases of metastatic disease in the subject. Periodic infusion or continuous infusion of the compound or pharmaceutical composition could also augment other standard cancer therapies for different cancers, particularly if the cancer has become metastatic.

Appropriate subjects for the methods provided herein include, without limitation, humans diagnosed or suspected of having a cancer (e.g., myosarcoma) or a skeletal muscle disorder (e.g., muscular dystrophy). Preferably, the subject is a human patient. More preferably, the subject has a disease (e.g., cancer, skeletal muscle disorder), has a genetic predisposition to such a disease, and/or exhibits a symptom of such a disease (e.g., skeletal muscle pathology, muscle atrophy, cachexia, skeletal muscle myopathy, dysfunctional autophagy, enhanced degradation of muscle proteins, reduced protein synthesis in skeletal muscle). As used herein, the terms “subject” and “patient” are used interchangeably herein and refer to both human and nonhuman animals. The term “nonhuman animals” of the disclosure includes all vertebrates, mammals and non-mammals, such as nonhuman primates, sheep, dog, cat, horse, cow, chickens, amphibians, reptiles, and the like. It will be apparent that compounds and pharmaceutical compositions described herein are effective at such alleviating conditions in subject other than humans, for example and not by way of limitation, domesticated animals, livestock, and horses.

Also provided are methods for protecting a subject from developing a cancer or from metastasis of an existing cancer or a skeletal muscle disorder such as a muscle wasting disease. In exemplary embodiments, a method of protecting a subject comprises administering to the subject a compound or pharmaceutical composition of the invention. Appropriate subjects for such methods include, without limitation, humans at high risk of developing a myosarcoma, a metastatic cancer, or a skeletal muscle disorder.

As used herein, the term “skeletal muscle disorder” refers to any condition characterized by muscle weakness and/or loss of muscle tissue and is characterized by or associated with a deregulated Hippo pathway. Suitable skeletal muscle disorders include, but are not limited to, myopathy, muscular dystrophy, myotonic dystrophy, and chronic obstructive pulmonary disorder. In some embodiments, the disease is muscular dystrophy, including but not limited to, Becker muscular dystrophy, Duchenne muscular dystrophy, Emery-Dreifuss muscular dystrophy, Facioscapulohumoeral muscular dystrophy, Myotonia congentia, and myotonic dystrophy. Preferably, the muscular dystrophy is Duchenne muscular dystrophy.

As used herein, the term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, hut such cells may exist alone within a subject, or may circulate in the blood stream as independent cells, such as leukemic or lymphoma cells. Preferably, the cancer is characterized by or associated with disruption of the Hippo signaling pathway. Suitable examples for cancer as used herein include, but are not limited to, sarcoma is rhabdomyosarcoma, synovial sarcoma, alveolar soft part sarcoma, liposarcoma, and osteosarcoma. Other cancers include, without limitation, NSCL, pancreatic, head and neck, colon, ovarian or breast cancers, or Ewing's sarcoma. However, cancers that may be treated by the methods described herein include lung cancer, bronchioloalveolar cell lung cancer, bone cancer, skin cancer, cancer of the head or neck, cutaneous or intraocular melanoma, uterine cancer, ovarian cancer, rectal cancer, cancer of the anal region, stomach cancer, gastric cancer, uterine cancer, carcinoma of the fallopian tubes, carcinoma of the endometrium, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, cancer of the bladder, cancer of the ureter, carcinoma of the renal pelvis, mesothelioma, hepatocellular cancer, biliary cancer, cancer of the kidney, renal cell carcinoma, chronic or acute leukemia, lymphocytic lymphomas, neoplasms of the central nervous system (CNS), spinal axis tumors, brain stem glioma, glioblastoma astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions. Also included within this definition is the term “solid tumor disease.” As used herein, the term “solid tumor disease” refers to those conditions, such as cancer, that for an abnormal tumor mass, such as sarcomas, carcinomas, and lymphomas. Suitable examples of solid tumor diseases include, but are not limited to, non-small cell lung cancer (NSCLC), neuroendocrine tumors, thyomas, fibrous tumors, mCRC, and the like.

In exemplary embodiments, the cancer is a “sarcoma,” As used herein, the term “sarcoma” includes malignant tumors of mesodermal connective tissue, e.g., tumors of bone, fat, and cartilage. Examples of sarcomas include, but are not limited to, rhabdomyoma, fibroma, lipoma, teratoma, Kaposi's sarcoma, fibrosarcoma, rhabdomyosarcoma, myxosarcoma, liosarcoma, chondrosarcoma, osteogenic sarcoma or chordosarcoma, liposarcoma, angiosarcoma, leiomysarcoma, Ewing's sarcoma, endotheliosardcoma, lympangiosarcoma, synoviosarcoma or mesothelisosarcoma. One would consider the method of the present invention to be a success if tumor growth were halted, reversed, or inhibited. Preferably, growth or spread of a tumor or metastatic tumor is inhibited by at least 25%. More preferably, growth or spread of a tumor or metastatic tumor is inhibited by at least 50% (e.g., at least 50%, 60%, 70%, 80%, 90%, 95%, 99%). In other cases, a method of the present invention is deemed to be a success if administration of a compound or pharmaceutical composition described herein induces myogenic differentiation of a sarcoma cell.

As used herein, the terms “Hippo pathway” and “Hippo signaling pathway” refer to asignaling pathway that utilizes serine/threonine protein kinases and adaptor proteins to regulate the expression of downstream targets that are regulators of cell cycle, apoptosis, and differentiation. Canonical Hippo signaling involves components such as mammalian Ste20-like serine/threonine kinases 1/2 (MST1/2), large tumor suppressor 1/2 serine/threonine protein kinases (LATS1/2), transcriptional co-activators Yes associated protein (YAP) and transcriptional co-activator with PDZ-binding motif (TAZ; also known as WWTR1). In response to upstream signals, MST1/2 are activated by phosphorylation. Phosphorylated MST1/2 complex with the scaffolding protein SAV then activates LATS1/2 kinases by phosphorylation. Activated LATS1/2, associated with their co-activator Mps-one binder 1 (MOB1), hyperphosphorylate YAP/TAZ on different sites. When the Hippo pathway is inactive or disrupted, YAP/TAZ are not phosphorylated by LATS1/2, allowing the transcriptional co-activators YAP/TAZ to accumulate in the nucleus which can result in the transcription of specific target genes involved in cell cycle, apoptosis and differentiation control.

As used herein, the term “interfering molecule” refers to any molecule that is capable of “interfering” with a molecule, such as signal transduction molecule, thereby modulating an intracellular signaling pathway (e.g., turn on or off). In preferred embodiments, the “interfering molecule” is capable of modulating the Hippo signaling pathway by interfering with the function of PAX3-FOX-1, RASSF4, MST1, LATS1, LATS2, YAP and/or TAZ. Examples of suitable interfering molecules include, but are not limited to, small molecules, peptides, polypeptides, cross-linking (hydrocarbon stapling) polypeptides, antisense RNAs, small temporal RNAs, cDNAs, dominant-negative forms of molecules, protein kinase inhibitors, combinations thereof, and the like.

In exemplary embodiments, an interfering molecule appropriate for use according to the methods provided herein is a member of the porphyrin family. Porphyrins are aromatic heterocyclic cyclic molecules composed of four modified pyrrole units interconnected at a carbon atoms via methine bridges. Exemplary porphyrins include, without limitation, protoporphyrin IX (PPIX), hematoporphyrin (HP), verteporfin (VP), and derivatives thereof. These porphyrin compounds were identified in a screen as capable of disrupting physical interactions between Yki/YAP and transcriptional coactivator TEAD/TEF transcription factors (Liu-Chittenden et al., Genes & Dev. 26:1300-1305 (2012)). Verteporfin is commercially available as an FDA-approved pharmaceutical: VISUDYNE® (verteporfin for injection), which is a 1:1 mixture of two verteporfin regioisomers. Hematoporphyrin (also known as 8,13-Bis(1-hydroxyethyl)-3,7,12,17-tetramethyl-21H,23H-porphine-2,18-dipropionic acid) is commercially available from chemical vendors such as Sigma-Aldrich. In some cases, a porphyrin compound is lipid-modified, lyophilized, and/or reconstituted in a pharmaceutically acceptable excipient or carrier for use as pharmaceutical composition. For example, VISUDYNE® is a lipid-modified, lyophilized preparation that is reconstituted prior to administration to a subject in need thereof.

As used herein, the term “cross-linking polypeptide” refers to those stably cross-linked polypeptides that comprise at least two modified amino acids (a process termed “hydrocarbon stapled”) that aid in conformationally bestowing the native secondary structure of that polypeptide. For example, cross-linking a polypeptide predisposed to have an alpha-helical secondary structure can constrain the polypeptide to its native alpha-helical conformation (see, e.g., U.S. Patent Application Publication No. 20090176964, filed Jul. 30, 3008 in the name of Loren D. Walensky and US Patent Application Publication No. 20050250680 filed Nov. 5, 2004 in the name of Loren D. Walensky). The constrained secondary structure can increase resistance of the polypeptide to proteolytic cleavage and also increase hydrophobicity. Such polypeptides can also penetrate the cell membrane (e.g., through an energy-dependent transport mechanism, e.g., pinocytosis). Accordingly, the cross-linked polypeptides envisioned herein can have improved biological activity relative to a corresponding uncrosslinked polypeptide. For example, a cross-linked polypeptide of the present disclosure may comprise an alpha-helical domain of a Hippo family member polypeptide (e.g., PAX3-FOXO1, RASSF4, MST1, LATS1, LATS2, YAP, TAZ, etc.) which can bind to it counterpart protein and modulate the Hippo pathway (e.g., in the case of cancer, turn on the Hippo pathway to allow for cell growth inhibition).

As used herein, the term “ameliorate” refers to the ability to make better, or more tolerable, a disease, such as cancer or skeletal muscle disorders. The term “prevent” refers to the ability to keep a disease, such as cancer or skeletal muscle disorders, from happening or existing. The term “treating” refers to the caring for, or dealing with, a cancerous condition or skeletal muscle disorder either medically or surgically. Also within the scope of the term “treating” is the acting upon a subject with cancer or skeletal muscle disorder with some agent, such as an interfering molecule, to improve or alter the cancerous condition.

As used herein, the terms “modulate” and “modulating activity” refer to up-regulating, down-regulating, or otherwise altering any one or more of the activities which a cell or a signaling pathway component is capable of performing such as, but not limited to, increasing or decreasing the role or extent to which a given component performs its function or modifying the nature of the function which a given component performs. For example, modulating the activity of a Hippo pathway component can include interfering with kinase activity by competition for a substrate or by an allosteric mechanism, or interfering with enzyme activation such as by altering a phosphorylation state. In some cases, modulating the activity of a signaling pathway or signaling pathway component is associated with quantitative differences between two states (e.g., allowing a signaling pathway to be turned on or off), preferably referring to at least statistically significant differences between the two states.

In an alternative embodiment of any of the above uses, the present disclosure also encompasses the use of a combination of at least one interfering molecule in combination with another cytotoxic, chemotherapeutic or anti-cancer agent(s), or compounds that enhance the effects of such agents, for the manufacture of a medicament for the treatment of cancer in a subject in need thereof, wherein each inhibitor or agent in the combination can be administered to the subject either simultaneously or sequentially. In this context, the “other anti-cancer agent or agent that enhances the effect of such an agent” will be dependent on the type(s) of cancer the subject is suffering from, and hence can be readily determined by one skilled in the art at the time of administration. Provided below are examples of suitable chemotherapeutic, cytotoxic, and/or anti-cancer agents which are within the scope of the present disclosure. This list is not intended to be limiting in any way.

In the context of this disclosure, other cytotoxic, chemotherapeutic or anti-cancer agents, or compounds that enhance the effects of such agents, include, but are not limited to: alkylating agents or agents with an alkylating action, such as cyclophosphamide (CTX; e.g., CYTOXAN™, chlorambucil (CHL; e.g., LEUKERAN™), cisplatin (C is P; e.g., PLATINOL™) busulfan (e.g., MYLERAN™), melphalan, carmustine (BCNU), streptozotocin, triethylenemelamine (TEM), mitomycin C, and the like; anti-metabolites, such as methotrexate (MTX), etoposide (VP16; e.g., VEPESID™), 6-mercaptopurine (6 MP), 6-thiocguanine (6TG), cytarabine (Ara-C), 5-fluorouracil (5-FU), capecitabine (e.g., XELODA™), dacarbazine (DTIC), and the like; antibiotics, such as actinomycin D, doxorubicin (DXR; e.g., ADRIAMYCIN™), daunorubicin (daunomycin), bleomycin, mithramycin and the like; alkaloids, such as vinca alkaloids such as vincristine (VCR), vinblastine, and the like; and other antitumor agents, such as paclitaxel (e.g., TAXOL™) and pactitaxel derivatives, the cytostatic agents, glucocorticoids such as dexamethasone (DEX; e.g., DECADRON™) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g., ETHYOL™), dactinomycin, mechlorethamine (nitrogen mustard), streptozocin, cyclophosphamide, lomustine (CCNU), doxorubicin lipo (e.g., DOXIL™), gemcitabine (e.g., GEMZAR™), daunorubicin lipo (e.g., DAUNOXOME™), procarbazine, mitomycin, docetaxel (e.g., TAXOTERE™, aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

With regards to radiation or a radiopharmaceutical, the source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. Parameters of adjuvant radiation therapies are, for example, contained in International Patent Publication WO 99/60023.

In another embodiment, provided herein is a method for detecting variation in the expression of Hippo-associated proteins, including PAX3-FOXO1, RASSF4, MST1, LATS1, LATS2, YAP and/or TAZ. The method can comprise or consist essentially of determining that level of PAX3-FOXO1, RASSF4, MST1, LATS1, LATS2, YAP and/or TAZ or determining specific alterations in the expressed product. Obviously, this sort of assay has importance in the diagnosis of diseases such as skeletal muscle disorders and cancers as described herein. Furthermore, subcellular localization and/or posttranslational modification to proteins such as RASSF4 may also he important for diagnostic and/or therapeutic purposes.

The biological sample can be any tissue or fluid. Various embodiments include cells of muscle, brain, lung, liver, spleen, kidney, lymph node, small intestine, blood cells, pancreas, colon, stomach, cervix, breast, endometrium, prostate, testicle, ovary, skin, head and neck, esophagus, oral tissue, bone marrow and blood tissue. Other embodiments include fluid samples such as peripheral blood, lymph fluid, ascites, serous fluid, pleural effusion, sputum, cerebrospinal fluid, lacrimal fluid, stool, or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given subject with a statistically significant reference group of normal patients and patients that have pathologies associated with expression of PAX3-FOXO1, RASSF4, MST1, LATS1, LATS2, YAP and/or TAZ. In this way, it is possible to correlate the amount or kind of PAX3-FOXO1, RASSF4, MST1, LATS1, LATS2, YAP and/or TAZ detected with various clinical states.

Alterations of a gene include deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of Hippo produced, both by altering the transcription of the gene or in destabilizing or otherwise altering the processing of either the transcript (mRNA) or protein.

A. Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should he spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described herein.

B. Separation Methods

Generally, it can be desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al 1989.

Alternatively, chromatographic techniques may he employed to effect separation. There are many kinds of chromatography which may he used in the present invention: adsorption, partition, ion-exchange and molecular sieve, and many specialized techniques for using them including column, paper, thin-layer and gas chromatography (Freifelder, 1982).

C. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

D. Kit Components

All the essential materials and reagents required for detecting and sequencing PAX3-FOXO1, RASSF4, MST1, LATS1, LATS2, YAP and/or TAZ and variants thereof may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification. Such kits also generally will comprise, in suitable means, distinct containers for each individual reagent and enzyme as well as for each primer or probe.

E. Quantitative or Semi-Quantitative PCR

Reverse transcription (RT) of RNA to cDNA followed by quantitative or semi-quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

F. Immuno-Diagnostic Methods

Antibodies may be used in characterizing the PAX3-FOXO1, RASSF4, MST1, LATS1, LATS2, YAP and/or TAZ content of healthy and diseased tissues, through techniques such as ELISAs and Western blotting. This may provide a screen for the presence or absence of malignancy or as a predictor of future cancer. For example, anti-PAX3-FOXO1, anti-RASSF4, anti-MST1, anti-LATS1, anti-LATS2, anti-YAP and/or anti-TAZ antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation. Following formation of specific immunocomplexes between the test sample and the bound antibody, and subsequent washing, the occurrence and even amount of immunocomplex formation may be determined by subjecting same to a second antibody having specificity for Hippo that differs the first antibody. Appropriate conditions preferably include diluting the sample with diluents such as BSA, bovine gamma globulin (BGG) and phosphate buffered saline (PBS)/Tween™. These added agents also tend to assist in the reduction of nonspecific background. The layered antisera is then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween™ or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease, alkaline phosphatase, glucose oxidase, or (horseradish) peroxidase-conjugated anti-human IgG for a period of time and under conditions which favor the development of immunocomplex formation (e.g., incubation for 2 hr at room temperature in a PBS-containing solution such as PBS/Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity tar the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

The disclosure may be better understood by reference to the following non-limiting Examples, which are provided as exemplary of the disclosure. The following examples are presented in order to more fully illustrate the preferred embodiments of the disclosure and should in no way be construed, however, as limiting the broad scope of the disclosure.

Pharmaceutical Compositions

In another aspect, provided herein are pharmaceutical compositions comprising a molecule capable of modulating, interfering with, or disrupting Hippo pathway signal transduction, or a pharmaceutically acceptable salt thereof, in combination with a pharmaceutically acceptable carrier.

The term “pharmaceutically acceptable salt” refers to salts prepared from pharmaceutically acceptable non-toxic bases or acids. When a compound described for use according to a method provided herein is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When an interfering molecule of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The pharmaceutical compositions of the present disclosure comprise an interfering molecule (including pharmaceutically acceptable salts thereof) as active ingredients, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above, and are described herein. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the pharmaceutical compositions represented by the interfering molecule (including pharmaceutically acceptable salts of each component thereof) described herein can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an interfering molecule described herein (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation. The term “therapeutically effective amount” of a pharmaceutical composition is an amount which is sufficient for the desired pharmacological effect.

Thus, the pharmaceutical compositions of this invention may include a pharmaceutically acceptable carrier and an interfering molecule described herein (including pharmaceutically acceptable salts of each component thereof). An interfering molecule described herein (including pharmaceutically acceptable salts of each component thereof) can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

In exemplary embodiments, a pharmaceutical composition comprises a combination of an interfering molecule described herein and another agent such as an anti-cancer agent. Preferably, the anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Generally, unit dosage forms may contain between from about 1 mg to about 2 g of the active ingredient, typically 10 mg, 15 mg, 20 mg, 25 mg, 50 mg, 100 mg, 200 mg, 300 mg, 400 mg, 500 mg, 600 mg, 800 mg, or 1000 mg. In exemplary embodiments, an interfering molecule or compound is administered in a 15 mg dosage units. In some cases, the interfering compound is provided in a pharmaceutical formulation at a dose between about 1 mg/kg to about 200 mg/kg (e.g., about 1 mg/kg, 10 mg/kg, 15 mg/kg, 25 mg/kg, 50 mg/kg, 75 mg/kg, 100 mg/kg, 150 mg/kg, 200 mg/kg).

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use such as, for example, an aerosol, cream, ointment, lotion, dusting powder, or the like. Further, the compositions can be in a form suitable for use in transdermal devices. These formulations may be prepared utilizing a combination of an interfering molecule described herein (including pharmaceutically acceptable salts of each component thereof) via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an interfering molecule described herein (including pharmaceutically acceptable salts of each component thereof) may also be prepared in powder or liquid concentrate form.

Dosage levels for the interfering molecules and/or additional compounds such as cytotoxic agents, chemotherapeutic agents and the like will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

Articles of Manufacture

In another aspect, provided herein are unit dosage forms comprising a pharmaceutical composition described herein.

In a further aspect, provided herein are kits comprising an assembly of materials and reagents appropriate for detecting and/or sequencing PAX3-FOXO1, RASSF4, MST1, LATS1, LATS2, YAP and/or TAZ and variants thereof. Generally, such kits will comprise preselected primers and probes. In some cases, kits provided herein will additionally comprise reagents and enzymes suitable for amplifying nucleic acids such as, without limitation, polymerases (RT, Taq, Sequenase™, etc.), deoxynucleotides, and buffers to provide the necessary reaction mixture for amplification. Such kits also generally will comprise, in suitable means, distinct containers for each individual reagent and enzyme as well as for each primer or probe.

Articles “a” and “an” are used herein to refer to one or to more than one (i.e., at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

The disclosure may be better understood by reference to the following non-limiting Examples, which are provided as exemplary of the disclosure. The following examples are presented in order to more fully illustrate the preferred embodiments of the disclosure and should in no way be construed, however, as limiting the broad scope of the disclosure. Therefore, the scope of the appended claims should not be limited to the description of the embodiments contained herein.

### EXAMPLES

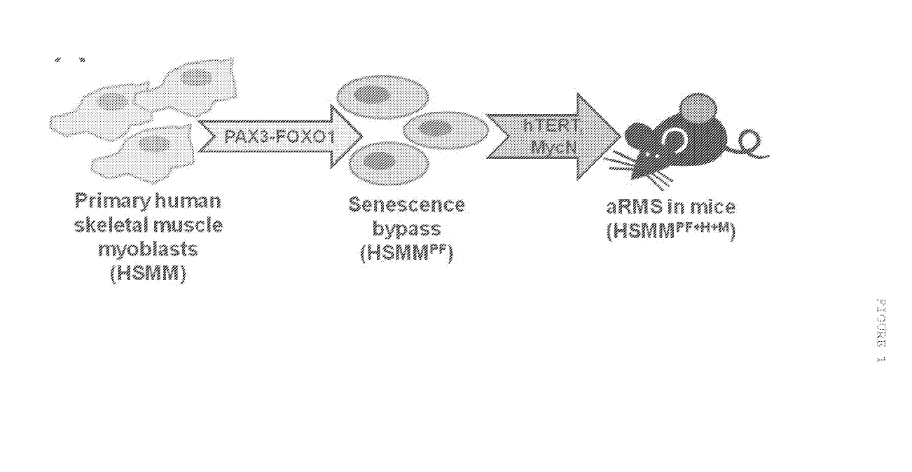

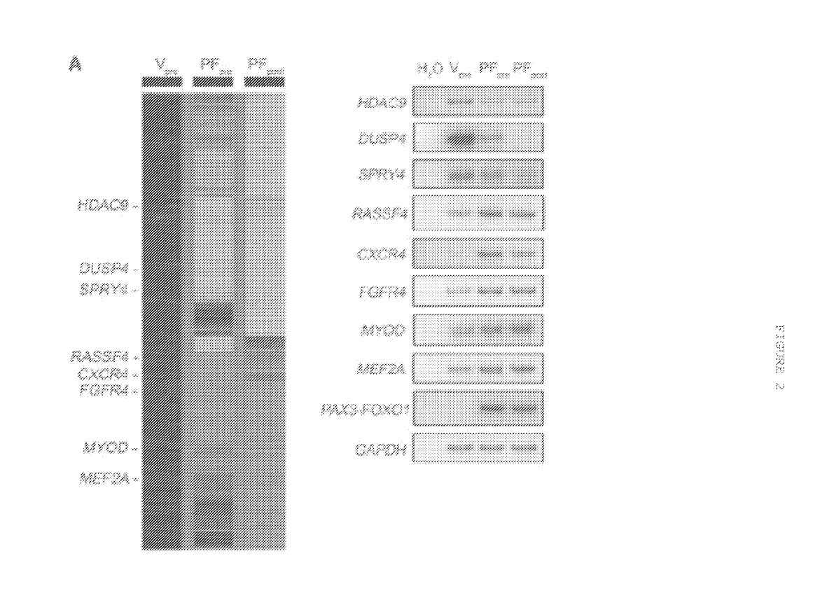

As described in the following Examples, the Inventors established an in vivo model for studying aRMS, the MST1/Hippo pathway, and the roles of MST1 and RASSF4 in cellular proliferation and tumorigenesis. Taking the complementary approach of modeling aRMS from primary (non-malignant) human skeletal muscle myoblasts (HSMMs), the Inventors developed a model allows for the study of aRMS initiation and step-wise progression in human cells and for large-scale, high-throughput screening and computational modeling for new therapeutic targets in vivo. In particular, the sequential expression in HSMMs of PAX3-FOXO1, followed by the catalytic subunit of telomerase (hTERT), followed by the MycN oncogene (model termed HSMM^{PF+H+M}) transformed HSMMs in vitro and produced xenografts in vivo that histologically mimic human aRMS (FIG. 1). Using this model, the inventors have discovered that the evolutionarily conserved Hippo tumor suppressor pathway is silenced in aRMS. Results show that this pathway is tightly regulated by a PAX3-FOXO1 transcriptional target known as RASSF4, suggesting that reactivation of the Hippo pathway will block tumorigenesis and provide a novel therapeutic strategy for aRMS.

### Example 1

### Identification of RASSF4 as a PAX3-FOXO1 Target

The Inventors previously demonstrated that early introduction of PAX3-FOXO1 contributes to HSMM bypass of cellular senescence, and also primes cells for aRMS tumorigenesis (Linardic et al., Cancer Res. 2007 Jul. 15; 67(14):6691-9). To understand the discrete molecular steps involved in aRMS tumorigenesis, a genetically defined model of aRMS was constructed based on the sequential introduction of a defined set of oncogenic cDNAs (PAX3-FOXO1, hTERT, MycN) into HSMMs. When implanted in immunodeficient mice, these transformed cells (abbreviated herein as HSMM^{PF+H+M}) produce xenografts whose morphology and histologic markers mimic aRMS (Naini S, et al. Defining the cooperative genetic changes that temporally drive alveolar rhabdomyosarcoma. Cancer Res. 2008; 68(23):9583-9588). The order of expression of the cDNAs is important for faithful generation of the model; most critically, PAX3-FOXO1 must be introduced first for the cells to transform in vivo. This suggests that PAX3-FOXO1 imparts critical cellular changes (genetic or epigenetic) that support subsequent tumorigenic steps and that investigation of these changes will provide needed insight into aRMS tumorigenesis.

During normal growth and development, the Hippo pathway provides tumor suppressor functions at the intersection of cell proliferation, differentiation, and apoptosis. Not surprisingly, malignancies have evolved to corrupt this pathway. MST/LATS loss or YAP overexpression lead to tumorigenesis in mouse models (9-14), demonstrating that Hippo pathway inhibition is sufficient for tumorigenesis. Identifying the mechanisms of Hippo pathway inactivation in human cancer will be paramount in finding ways to exploit this pathway therapeutically.

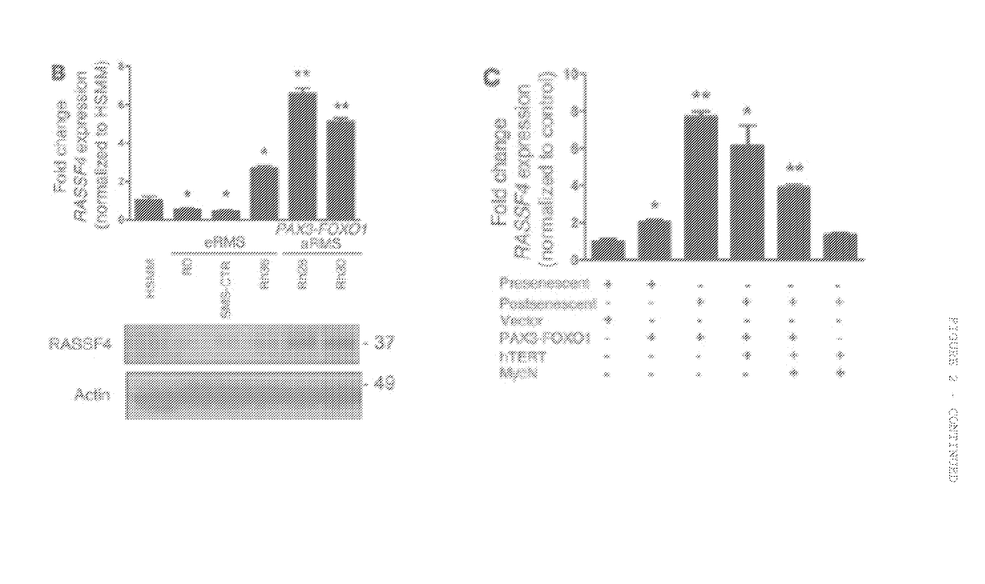

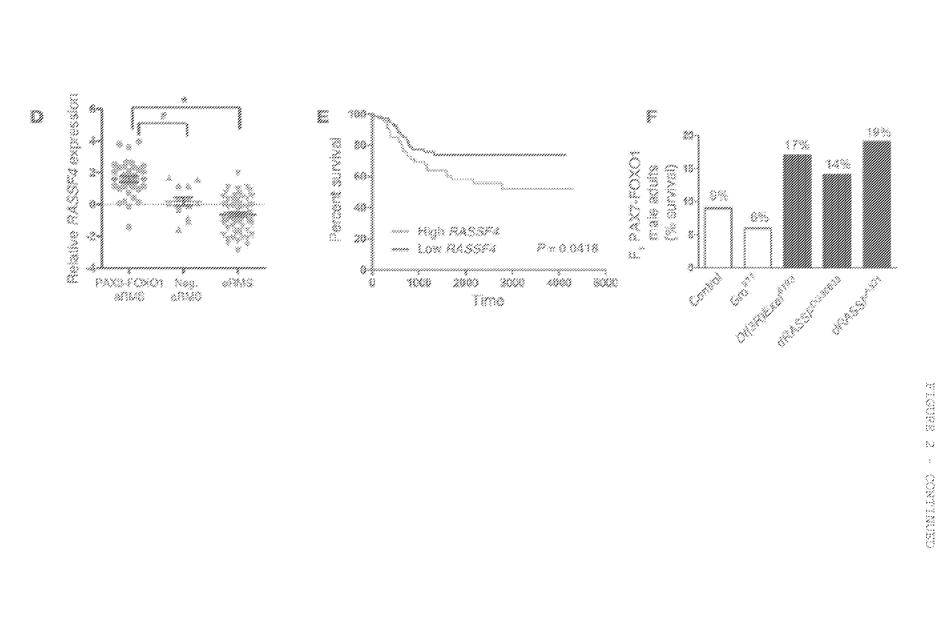

Global gene expression was compared for presenescent vector-expressing control HSMMs, presenescent PAX3-FOXO1-expressing HSMMs, and postsenescent PAX3-FOXO1-expressing HSMMs using Affymetrix mRNA microarray analysis. Gene expression profiles of all cells were normalized by robust multiarray average (RMA) and zero transformed against the average expression levels of the same probe sets of the vector-expressing control HSMMs. When compared to control myoblasts, the resulting ˜2600 significantly changed genes included PAX3-FOXO1 targets, as well as other many novel targets. One of these is the Ras Association Domain Family member RASSF4 (FIG. 2A, left). Upon further examination, we found that RASSF4 expression was markedly elevated in PAX3-FOXO1-positive human aRMS cells (data not shown due to space limitations) and tumors (FIG. 2C), with elevated RASSF4 levels associated with worse clinical outcome (FIG. 2D). These data suggested to us that we had identified RASSF4 as vital component of aRMS biology that had not been described previously.

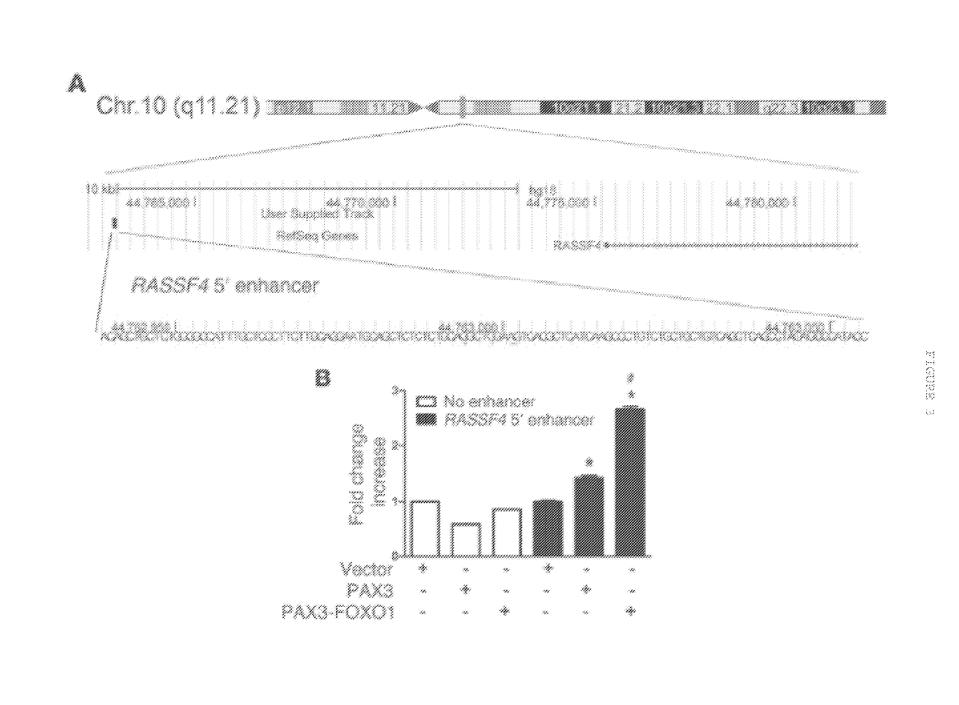

To determine the role of RASSF4 in aRMS, loss-of-function studies were performed. Using three independently-targeting RASSF4 shRNAs (FIG. 3A), it was found that RASSF4 suppression caused a profound growth arrest in both our genetic model of aRMS (FIG. 3B) and the Rh28 human aRMS cell line (not shown). RASSF4-knockdown cells also displayed. characteristics of senescence, including senescent morphology, beta-galactosidase staining (FIG. 3C), and p21 tumor suppressor induction (not shown). Based on these results, it was hypothesized that RASSF4 upregulation in aRMS is critical to promote proliferation and senescence evasion.

Next, a doxycycline (dox)-inducible system was created to conditionally express RASSF4 shRNAs. This system induces RASSF4 knockdown in the presence of dox and allowed for the assessment of the role of RASSF4 in aRMS xenografts in vivo. Inducible knockdown of RASSF4 significantly reduced tumor volume over time (FIGS. 3D-E), validating the pro-proliferative role of RASSF4 in vivo.

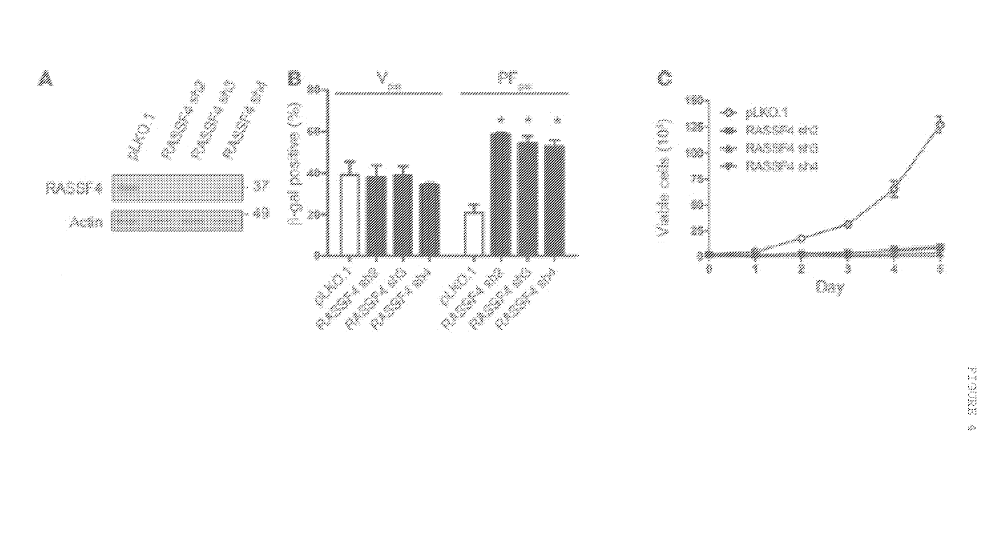

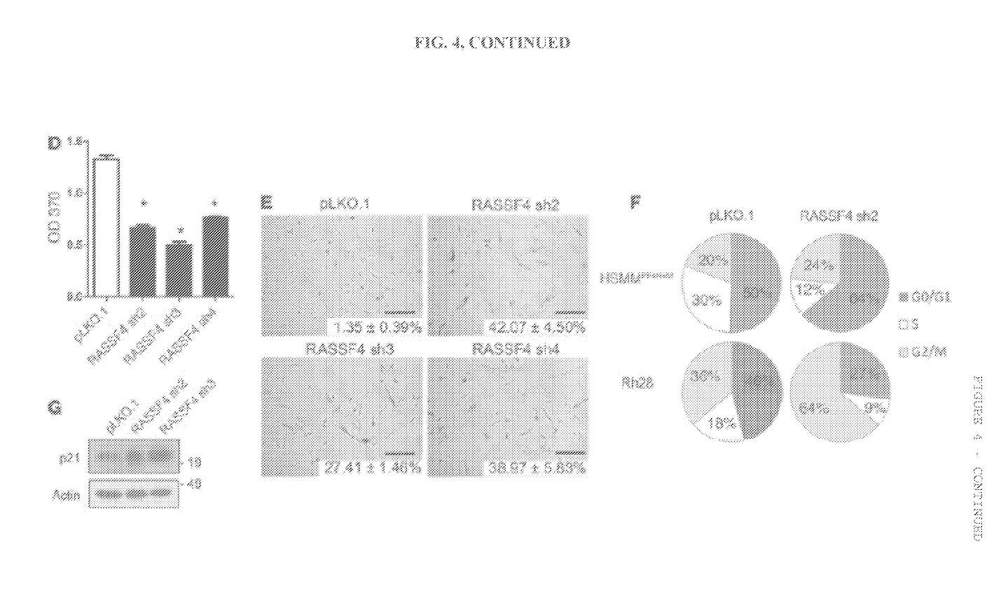

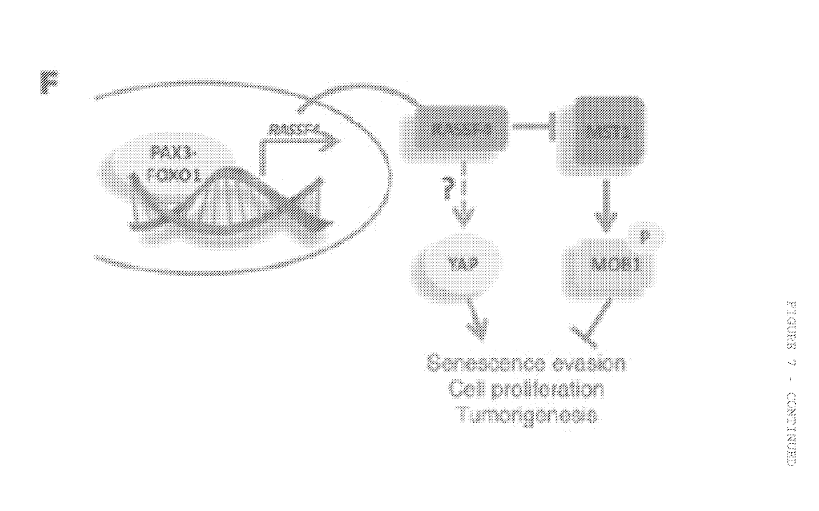

Since PAX-FOXO1 upregulated RASSF4 or dRASSF in two independent models of aRMS, we reasoned that RASSF4 influences human aRMS pathobiology. Therefore, we began to study the function of endogenous RASSF4 in HSMMs using RASSF4 RNAi loss of function. We generated five lentiviral shRNA plasmids independently targeting RASSF4. Three of these plasmids (RASSF4 sh2-4) were effective at suppressing RASSF4 protein expression in aRMS cells (FIG. 4A). As additional validation, we used semiquantitative RT-PCR to measure endogenous RASSF4 mRNA, which demonstrated shRNA-mediated suppression of endogenous RASSF4 in primary HSMMs (FIG. 9A).

To address the role of RASSF4 in PAX3-FOXO1-mediated senescence bypass, we stably expressed RASSF4 shRNAs in vector or PAX3-FOXO1-expressing HSMMs. To examine the effect of RASSF4 suppression on cellular senescence, we measured in situ β-gal activity, a biomarker of cellular senescence. In vector-expressing HSMMs, RASSF4 suppression did not change β-gal staining. In contrast, in PAX3-FOXO1-expressing cells, RASSF4 suppression induced a significant increase in the percentage of β-gal-positive cells, signifying that more cells were undergoing senescence (FIG. 4B). These data demonstrate that in the context of PAX3-FOXO1 expression, RASSF4 prevents the onset of cellular senescence.

Since RASSF4 suppression promotes senescence in PAX3-FOXO1-expressing HSMMs, we next examined the effect of reduced RASSF4 expression in our genetic model of aRMS, which is based on HSMMs expressing PAX3-FOXO1 (HSMM^{PF+H+M}, FIG. 2C). Stable expression of RASSF4 shRNAs in HSMM cells in culture caused a profound growth arrest (FIG. 4C) and decreased BrdU incorporation (FIG. 4D). Cells with reduced RASSF4 expression underwent substantial morphologic changes including enlargement, flattening, and increased granularity, reflecting senescence. In addition, β-gal staining was dramatically increased (FIG. 4E). These results suggest that, similarly to HSMMs expressing PAX3-FOXO1, our genetic model of aRMS also relies on RASSF4 expression to promote cell proliferation and inhibit senescence.

We next examined RASSF4-deficient cells for changes in their cell cycle profile. Depending on the cell type, cells were arrested at either G0/G1 or G2/M checkpoints. This suggests that in aRMS, RASSF4 can promote cell cycle progression at both checkpoints or that loss of RASSF4 causes an overall slowing of the cell cycle (FIG. 4F). Accordingly, we also observed elevation of p21 protein (FIG. 4G), which participates in both the G0/G1 and G2/M checkpoints. While we observed that RASSF4 knockdown cells were growth arrested for many days after transduction, they were ultimately not viable in culture after more than 1 week. However, the nonviable RASSF4 cells did not undergo apoptosis, as we observed no caspase 3 cleavage, annexin V positivity, or sub-G1 peak by flow cytometry (data not shown). These data suggest that RASSF4 promotes aRMS cell proliferation and viability by promoting cell cycle progression and evasion of cell senescence.

Together, in vitro studies point toward RASSF as a critical component of aRMS cell proliferation and viability. To investigate in vivo activity, we tested whether RASSF4 loss of function would alter tumorigenesis in an aRMS xenograft model. Since RASSF4 suppression caused growth arrest and loss of cell viability, we used a Tet-on lentiviral shRNA system to induce RASSF4 knockdown upon doxycycline (Dox) exposure. We used the PAX3-FOXO1-positive Rh28 cell line to establish this dox-inducible system, first recapitulating prior RASSF4 knockdown data as demonstrated by decreased BrdU incorporation and increased p21 protein (FIGS. 9B-E).

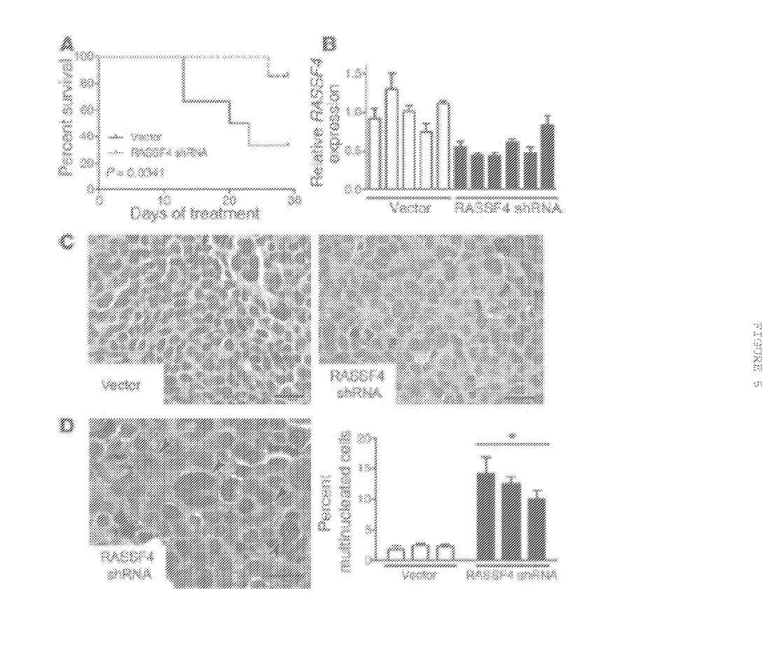

Next, we tested the effects of RASSF4 suppression in aRMS xenografts in vivo. With doxycycline treatment, Rh28 aRMS xenografts expressing RASSF4 shRNA exhibited a significant (P=0.0341) delay in reaching maximum tumor burden when compared with control (FIGS. 5A-B) Our examination of RASSF4-knockdown tumors for changes in the proliferative index using Ki67 immunostaining revealed no changes (data not shown). However, H&E-stained tumor sections revealed dramatic changes in cell morphology. We found that RASSF4-suppressed tumors contained large cells with increased cytoplasm and prominent nucleoli and an abundance of cells with large, irregularly shaped nuclei and multinucleated giant cells (FIGS. 5C-D). These cellular morphologies suggest mitotic defects and growth arrest (37), which could explain the decreased tumor growth in RASSF4-knockdown tumors. Based on these data, we concluded that, similarly to in vitro studies, RASSF4 serves a pro-proliferative role in aRMS in vivo.

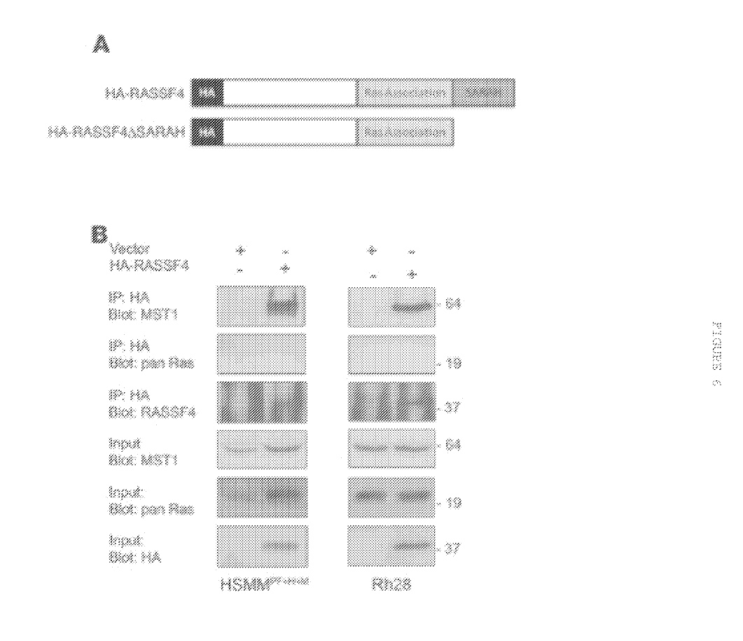

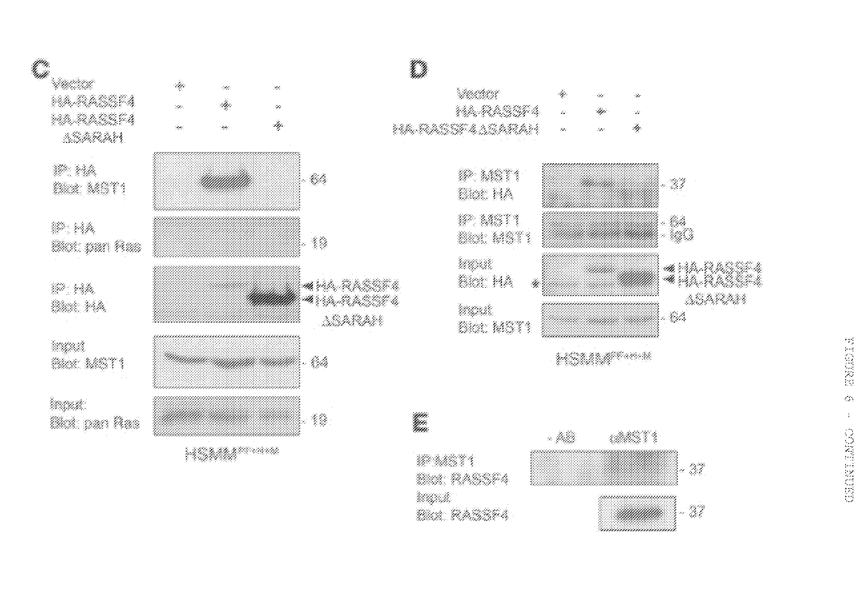

Having demonstrated that RASSF4 is pro-proliferative in aRMS both in vitro and in vivo, we next examined the RASSF4 signaling mechanism in aRMS cells. RASSF proteins are typically described as scaffolding molecules, regulating protein complex formation to coordinate signaling cascades. Consistent with this notion, RASSF4, which possesses no identifiable catalytic domains, contains a Ras-association domain and a carboxy-terminal Salvador/RASSF/Hippo (SARAH) domain (FIG. 6A). To identify proteins that associate with RASSF4, we immunopurified epitope-tagged RASSF4 protein complexes and probed for endogenous RASSF4-associating proteins. We were surprised to find no association of HA-RASSF4 with any of the canonical Ras isoforms (H-, K-, and N-Ras) in HSMM^{PF+H+M} cells (FIG. 6B, left). We observed identical results with the PAX3-FOXO1-positive Rh28 cell line (FIG. 6B, right). Therefore, under these conditions, RASSF4 does not associate with Ras.

RASSF4 also contains a carboxy-terminal SARAH domain (FIG. 6A), which was predicted to associate with SARAH domain-containing proteins of the Hippo pathway. SARAH domain hetero- and homodimerizations play critical roles in the regulation of the Hippo pathway at the level of MST kinases, which also contain SARAH domains. As other members of the RASSF family have been shown to interact with MST1, we tested whether RASSF4 does as well. In both HSMM^{PF+H+M} and Rh28 lysates, we found coprecipitated, endogenous MST1 with HA-RASSF4 (FIG. 6B). To determine whether the SARAH domain of RASSF4 regulated the association with MST1, we generated a RASSF4 construct lacking the SARAH domain (HA-RASSF4ΔSARAH) (FIG. 6A) and stably expressed this plasmid in HSMM^{PF+H+M} cells. Deletion of the SARAH domain from RASSF4 was sufficient for a loss of association with MST1 (FIG. 6C). Similarly to previous experiments, Ras was not found to co-precipitate with HA-RASSF4 or HA-RASSF4ΔSARAH (FIG. 6C). We then performed the reverse experiment by immunopurifying endogenous MST1 from aRMS cells and immunoblotting for copurified HA-RASSF4 or HA-RASSF4ΔSARAH. We observed a similar association between MST1 and HA-RASSF4, but not HA-RASSF4ΔSARAH (FIG. 6D). Additional studies were performed using an epitope-tagged RASSF4 SARAH domain to examine the sufficiency of RASSF4 interaction with MST1, but these experiments were inconclusive, as the SARAH domain was not expressed at detectable levels. This may be due to protein insolubility, which has been observed with the RASSF1 SARAH domain (Hwang et al., Proc Natl Acad Sci USA 104:9236-9241 (2007)). Last, we validated the endogenous MST1-RASSF4 protein complex using MST1 immunoprecipitation (FIG. 6E). Altogether, we found that in aRMS cells, RASSF4 associates with MST1, and this association is dependent on the RASSF4 SARAH domain.

As a core component of the Hippo signaling pathway, MST1 plays a critical role in promoting the phospho-relay cascade that results in growth arrest, differentiation, or apoptosis. Therefore, we hypothesized that RASSF4 associates with MST1 as an inhibitory complex to block activation of the Hippo pathway. If this hypothesis were true, then artificial activation of MST1 in aRMS cells should phenocopy RASSF4 loss. Therefore, we generated stable aRMS cell lines expressing wildtype MST1, kinase-dead MST1 (MST1K59R), or vector control. We found that MST1 expression in aRMS cells was not tolerated, as cells were not viable in culture beyond two to three passages. This effect was dependent on the kinase activity of MST1, since expression of MST1K9R had no effect on aRMS cell growth and proliferation in culture (data not shown). To test whether activation of the Hippo pathway through overexpression of MST1 resulted in senescence induction, as seen with RASSF4 knockdown, we performed a senescence assay on HSMM^{PF+H+M} cells expressing MST1, MST1K59R, or vector alone. Cells expressing MST1, but not MST1K59R, had significantly higher β-gal staining than did vector-expressing control cells (FIG. 6F), suggesting that MST1 signaling in aRMS cells is sufficient to cause growth arrest and senescence. To determine the effect of RASSF4 on the senescence-promoting activity of MST1, we generated HSMM^{PF+H+M} cell lines expressing either MST1 or MST1K59R in combination with HA-RASSF4 or HA-RASSF4ΔSARAH. Coexpression of MST1 with HA-RASSF4 blunted the senescence-inducing effects of MST1, but expression of MST1 with HA-RASSF4ΔSARAH, which cannot bind MST1, induced senescence similarly to cells expressing MST1 alone (FIG. 6F). Based on these data, we conclude that Hippo pathway signaling by MST1 overexpression induces growth arrest and senescence in aRMS cells. RASSF4 expression blocks this function of MST1, suggesting that RASSF4 may act as a Hippo pathway inhibitor. We next tested the hypothesis that RASSF4 inhibits MST1-mediated signaling in aRMS. As a readout for MST1 activation, we examined the phosphorylation status of LATS1 and MOB1. We performed these experiments in the context of nocodazole treatment, as MST1 has been shown to he activated under these conditions and to promote phosphorylation of LATS1 and MOB1 (Praskova et al., Curr. Biol. 18:311-321 (2008)).

Surprisingly, we observed a reduction in phosphorylated LATS1 in nocodazole-treated cells, which was similar between control and RASSF4-knockdown cells (FIG. 6G). Although we did not see changes in signaling to LATS1 in aRMS cells with RASSF4 loss, we did observe higher basal and nocodazole-induced phosphorylated MOB1. To determine whether these effects were due to MST1 signaling, we performed RASSF4 knockdown in cells expressing kinase-dead MST1 K59R. Again, we did not Observe a significant change in the phosphorylation of LATS1 in cells expressing MST1 K59R. (FIG. 6G). However, the expression of MST1 K59R, completely blocked the induction of phosphorylated MOB1 in RASSF4-knockdown cells. Further, MST1 K59R prevented the induction of p21 in RASSF4-deficient cells. To determine whether MST1 K59R, expression reversed the RASSF4-knockdown phenotype, we measured senescence induction and cell cycle progression. MST1 K59R expression ameliorated the senescence induction and cell cycle arrest caused by RASSF4 loss (FIGS. 5H and I). Based on these data, we conclude that aRMS cells promote senescence evasion through RASSF4-mediated suppression of MST1 signaling.

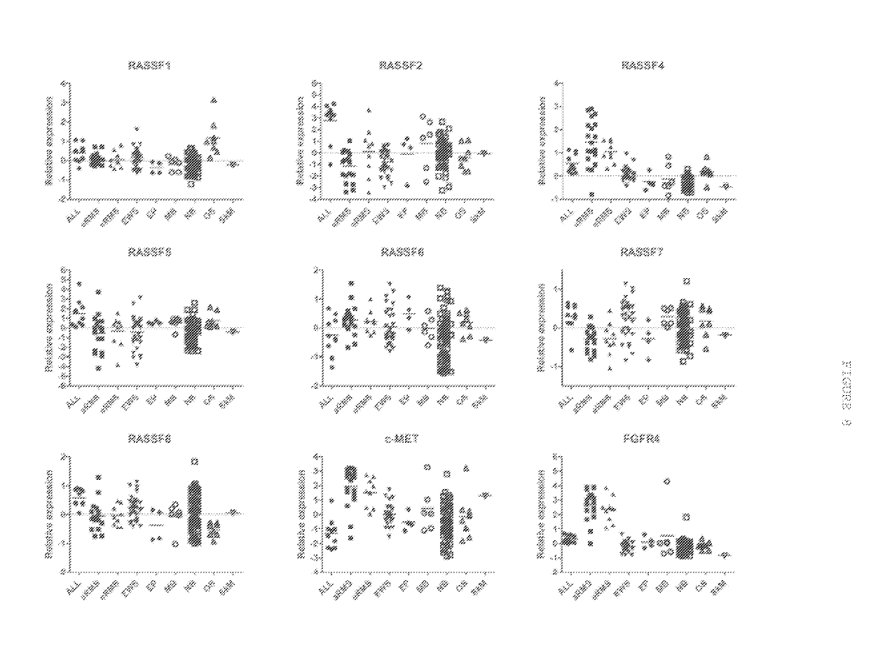

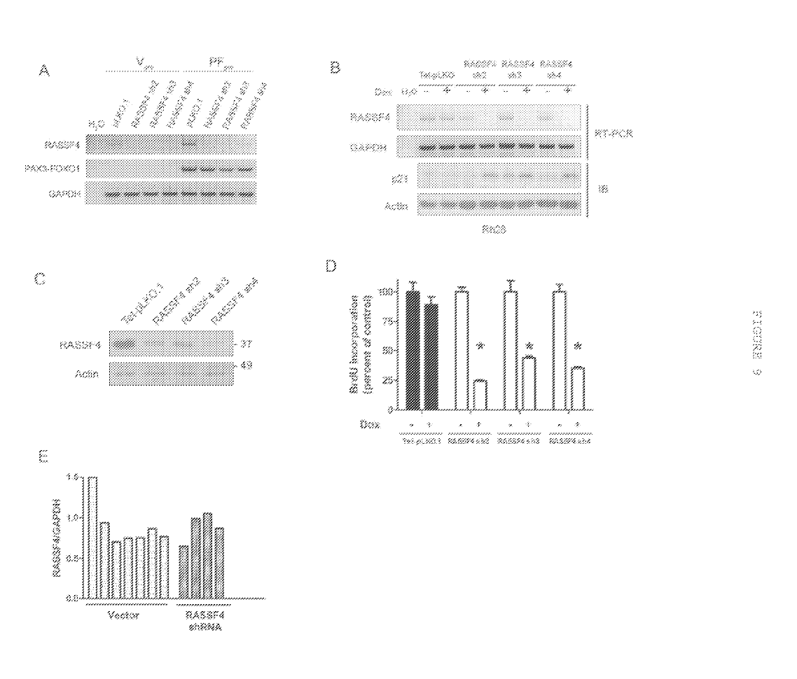

As described above, the expression of kinase-dead MST1 K59R did not impact aRMS cell growth or viability. However, even without the ability to activate downstream signaling, MST1 K59R can still be regulated like wild-type MST1 through phosphorylation by upstream kinases and caspase cleavage. Since endogenous phospho-MST1 levels and MST1 cleavage products were in low abundance in aRMS cells and were difficult to detect, we used cells expressing MST1K59R to boost MST1 expression, while maintaining cell viability. We stimulated cells with staurosporine (STS), a well-established activator of MST1, and examined the role of RASSF4 in MST1 phosphorylation and cleavage (Graves et al., J. Biol. Chem 276:14909-14915 (2001)). We used immunoblotting to measure STS treatment in MST1 expressing HSMM^{PF+H+M} cells and found that it led to increased phosphorylation of full-length MST1. In RASSF4-deficient cells, phospho-MST1 levels were higher in response to STS and also exhibited an approximately 35-kDa cleavage fragment (FIG. 10A). This is the reported size of the MST1 cleavage fragment believed to be regulated by caspases (Graves et al. (2001); Cheung et al., Cell 113:507-517 (2003); Praskova et al., Biochem J. 381:453-462 (2004)). We found that RASSF4-deficient. STSA-treated aRMS cells displayed increased cleaved caspase 3 species in cells expressing MST1 K59R (data not shown) or in cells with RASSF4 knockdown alone (FIG. 10B). In summary, RASSF4 may act as an endogenous inhibitor of the Hippo pathway in aRMS cells by preventing apoptotic signaling to caspase 3, blocking MST1 phosphorylation, and/or restraining MST1-mediated signaling pathways.

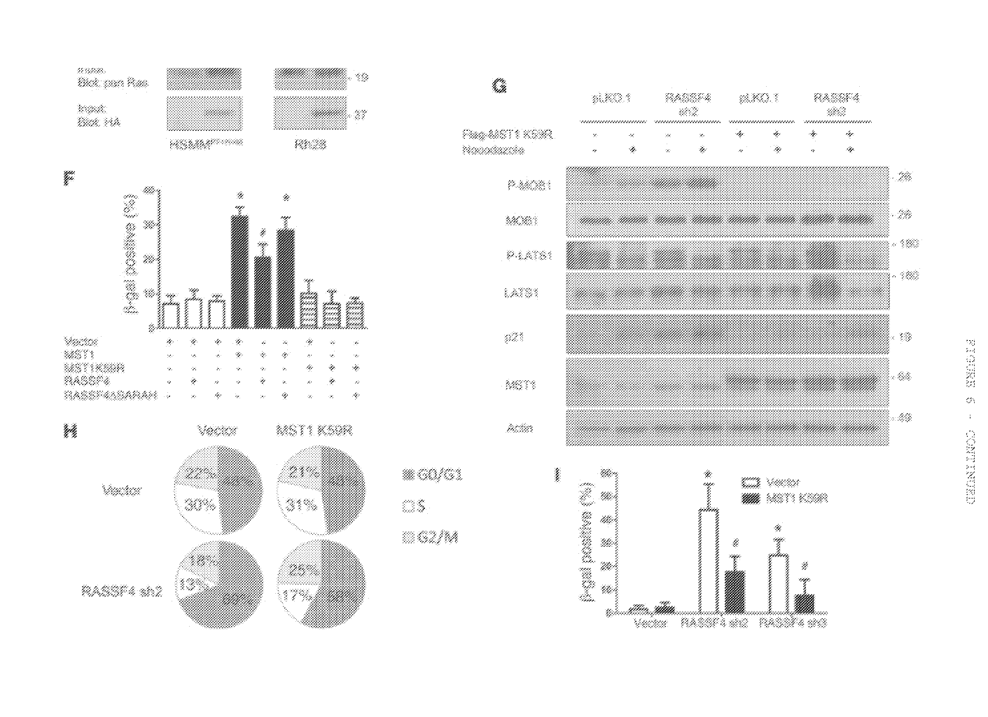

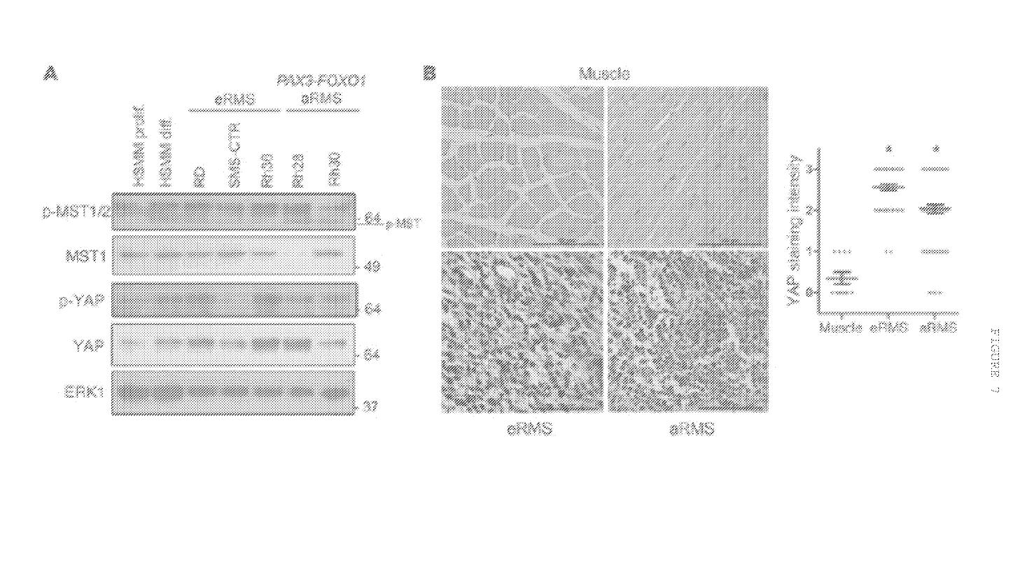

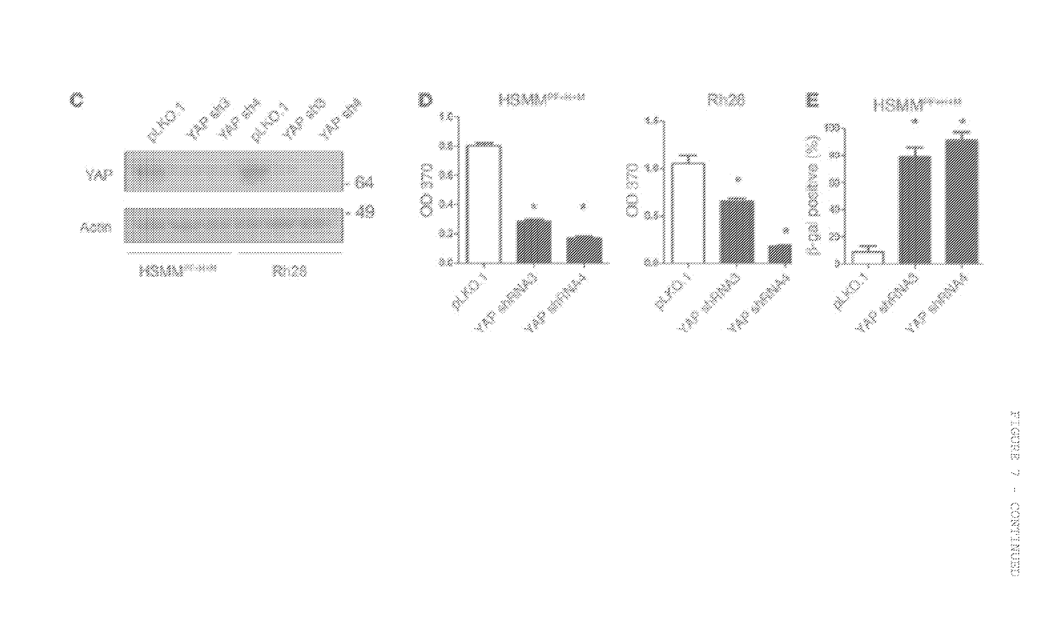

Hippo signaling is suppressed in aRMS. The PAX3-FOXO1/RASSF4/MST1 signaling axis identified in aRMS cells suggests that Hippo pathway suppression may play a critical role in aRMS biology. Since the Hippo pathway has not been examined in the context of RMS, we next examined the status of core Hippo signaling components in RIMS cell lines and tumors. First, we examined the levels of phospho-YAP, YAP, phospho-MST1/2, and total MST1 in RMS cell lines by immunoblot analysis. We compared these levels with primary, proliferating HSMMs and in vitro differentiated HSMMs. To examine phosphorylated MST1, we used commercially available antibodies against phospho-MST1/2 (MST1 T183, MST2 T180). The resulting immunoblot had a predominant immunoreactive band that was much larger than the predicted size of phosphorylated MST1/2 (FIG. 7A). A less dominant band was evident at the correct size (˜60 kDa) and showed variable abundance in the HSMM and RMS cell lines. The identity of the larger molecular weight band is not known, however, phosphorylated PAK1 (˜70 kDa) and phosphorylated MST1/2 are known to cross-react on immunoblots (Ribeiro et al., Mol. Cell 39:521-34 (2010); Lian et al., J. Immunol. 166:6349-57 (2001)). Phospho-YAP (S127) was induced with in vitro differentiation of myoblasts, as has been observed previously (Watt et al., Biochem Biophys Res Commun 393:619-624 (2010)). We observed that phospho-YAP levels were highly variable in RMS cell lines and did not correlate with histological subtype. However, when we examined total YAP, there appeared to be upregulation of YAP protein in many of RMS cell lines (FIG. 7A). Therefore, while phosphorylation of MST1 and YAP in RMS cell lines was variable, YAP protein was upregulated, suggestive of a role for YAP in RMS.

We then examined the status of YAP in human RMS tumors. Using an RMS tissue microarray (TMA), we performed immunohistochemistry on 356 tissue samples representing 130 individual RMS tumors (or normal muscle) for YAP protein expression. In cross sections of normal muscle, YAP was rarely detectable, and in longitudinal sections, YAP was only weakly stained in the cytoplasm (FIG. 7B). In both cases, there was no significant nuclear YAP staining, suggesting that in normal muscle, YAP was not playing an active, transcriptional role. Conversely, we found that both eRMS and aRMS samples showed high expression of cytoplasmic and nuclear YAP protein (representative samples, FIG. 6B). Therefore, similarly to other cancers, RMS malignancies may upregulate the YAP oncoprotein for tumor growth and survival.

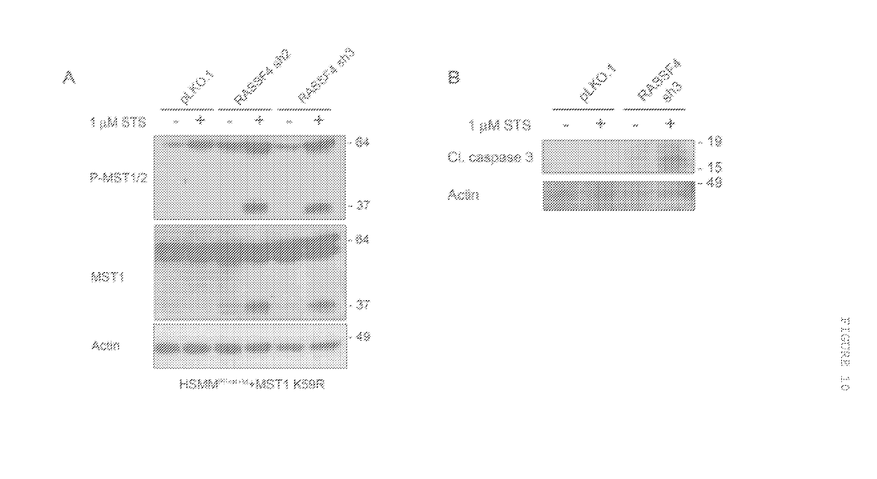

We next examined the role of YAP itself in aRMS cells. YAP-deficient aRMS cells were significantly less proliferative than control cells (FIGS. 7C-D) and displayed a dramatic increase in senescence-associated 1-gal staining (FIG. 7E). Since YAP has been shown to regulate transcriptional complexes, we examined the effect of YAP loss on the YAP target gene CTGF. Surprisingly, CTGF levels in aRMS cells were much lower than in nontransformed HSMMs (FIG. 11A). Additionally, YAP knockdown, while causing growth arrest and senescence in aRMS cells, had no effect on CTGF expression (FIG. 11B). Together, these data suggest that either CTGF is not a YAP target gene in aRMS cells, or that YAP functions in aRMS in a manner distinct from that observed in other human cancers.

Finally, we examined whether there was a connection between RASSF4 and YAP. Compared with control aRMS cells, phospho-YAP and total YAP protein levels in RASSF4-knockdown aRMS cells were significantly reduced. Since YAP protein is turned over by sequential phosphorylation and ubiquitin-mediated degradation (Zhao et al., Genes Dev. 24:72-85 (2010)), we attempted to pharmacologically block YAP protein turnover in RASSF4-deficient cells using MG132 or IC 261. Despite these treatments, phospho-YAP and total YAP levels were still lower in RASSF4-deficient cells (data not shown), suggesting alternative mechanisms of YAP regulation. When examined at the transcriptional level, we observed a decreasing trend in YAP mRNA levels that only reached significance with one RASSF4 shRNA. Since RASSF4 loss of function was causing growth arrest and senescence, we reasoned that these effects on YAP could be caused by the growth arrest itself and RASSF4 loss. Similarly, YAP S127A add-back experiments in RASSF4-knockdown cells did not fully reverse cell viability in RASSF4-deficient cells. Instead, cells expressing YAP S127A and RASSF4 shRNA had more pronounced mitotic defects (data not shown). Therefore, while we cannot identify a clear connection between RASSF4 and YAP in aRMS at this time, Hippo signaling is clearly dysregulated at multiple levels in RMS through RASSF4-MST1 and YAP-mediated mechanisms (FIG. 7F).

### Example 2

### In Vivo Tumor Treatment

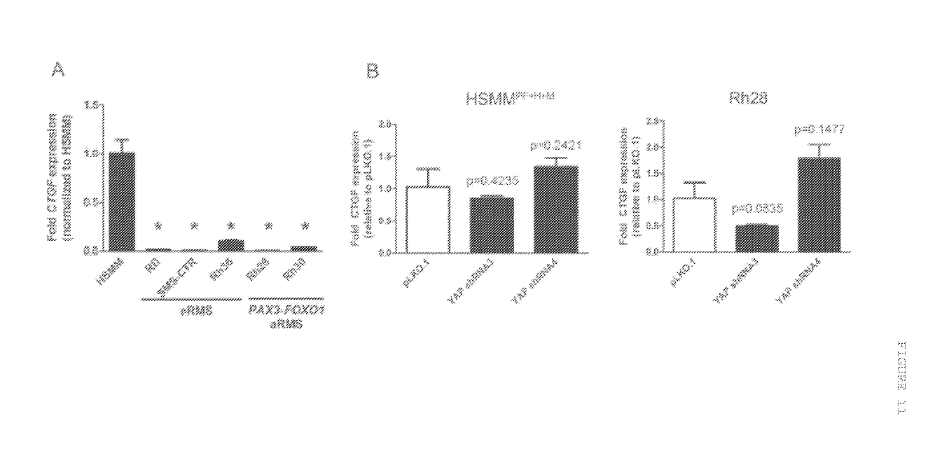

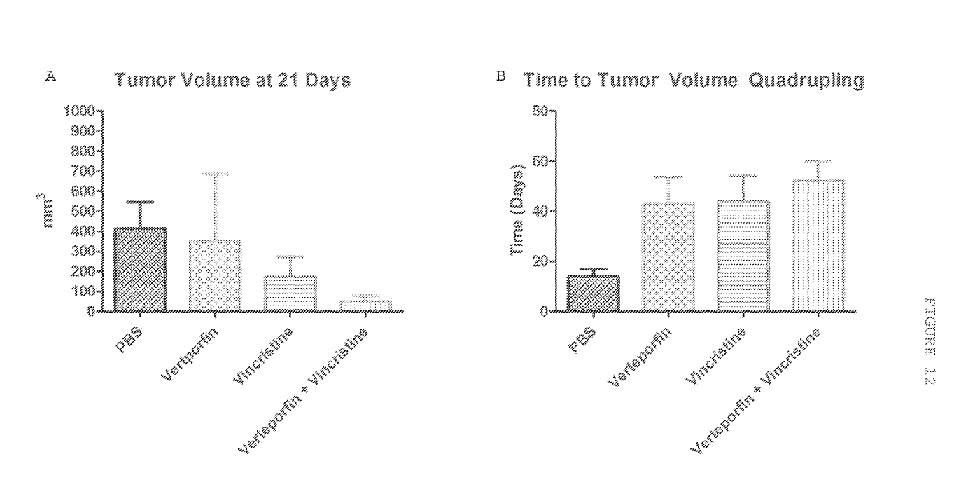

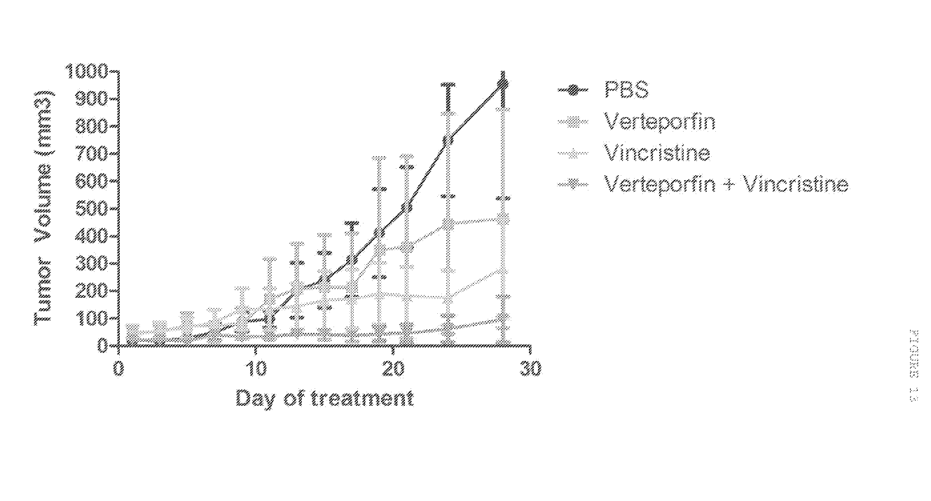

Alveolar rhabdomyosarcoma (aRMS) xenografts using Rh28 cells were subcutaneously implanted in SCID/beige mice (10 million cells per xenograft). When tumors were palpable, mice were treated with intraperitoneal treatment with either phosphate buffered saline (PBS, negative control), Verteporfin (100 mg/kg, 3 times per week), Vincristine (1 mg/kg, 1 time per week), or a combination of Verteporfin and Vincristine (Vincristine 1 mg/kg 1 time per week, Verteporfin 100 mg/kg 2 times per week). Mice were treated for three weeks and tumors were measured twice weekly. See FIG. 12 and FIG. 13.

We claim:

1. A method of treating a disease in a subject in need thereof, the method comprising administering to said subject a therapeutically effective amount of at least one compound capable of modulating at least one Hippo signaling pathway component, whereby administration of the at least one compound treats a disease or condition associated with a disrupted Hippo signaling pathway.

2. The method of claim 1, wherein the disease is selected from the group consisting of a cancer, skeletal muscle disorder, myopathy, muscular dystrophy, myotonic dystrophy, and chronic obstructive pulmonary disorder.

3. The method of claim 2, wherein said disease is muscular dystrophy.

4. The method of claim 3, wherein said muscular dystrophy is selected from the group consisting of Becker muscular dystrophy, Duchenne muscular dystrophy, Emery-Dreifuss muscular dystrophy, Facioscapulohumoeral muscular dystrophy, Myotonia congentia, and myotonic dystrophy.

5. The method of claim 4, wherein said muscular dystrophy is Duchenne muscular dystrophy.

6-7. (canceled)

8. The method of claim 1, wherein the subject is human.

9. The method of claim 1, wherein the at least one compound is a porphyrin selected from the group consisting of verteporfin, protoporphyrin IX, and hematoporphyrin, or a derivative thereof.

10. The method of claim 1, wherein the at least one compound is a cross-linked polypeptide or an antisense RNA molecule.

11. (canceled)

12. The method of claim 1, wherein the at least one compound modulates a Hippo signaling pathway component selected from the group consisting of RASSF4, MST1, PAX3-FOXO1, LATS1, LATS2, YAP, and TAZ.

13. The method of claim 1, further comprising administering a therapeutically effective amount of a chemotherapeutic agent to the subject.

14. (canceled)

15. The method of claim 1, further comprising exposing the subject to radiation.

16-18. (canceled)

19. A method of diagnosing a disease in a subject, the method comprising:
(a) detecting an expression level of a Hippo signaling pathway component in a biological sample of the subject; and
(b) comparing the detected expression level to an expression level of the Hippo signaling pathway component detected in a biological sample of a control subject having a known disease or condition associated with a disrupted Hippo signaling pathway.

20. The method of claim 19, wherein the Hippo signaling pathway component is selected from the group consisting of PAX3-FOXO1, RASSF4, MST1, LATS1, LATS2, YAP, and TAZ.

21. The method of claim 20, wherein the Hippo signaling pathway component is RASSF4.

22. The method of claim 20, wherein the Hippo signaling pathway component is MST1.

23. (canceled)

24. The method of claim 19, wherein detecting comprises measuring a nucleic acid or polypeptide level of at least one of PAX3-FOXO1, RASSF4, MST1, LATS1, LATS2, YAP or TAZ.

25. The method of claim 24, wherein measuring comprises a technique selected from the group consisting of polymerase chain reaction, immunohistochemistry, and ELISA.

26. The method of claim 19, wherein detecting comprises identifying a subcellular location of RASSF4.

27. The method of claim 19, further comprising evaluating the stability of a nucleic acid encoding PAX3-FOXO1, RASSF4, MST1, LATS1, LATS2, YAP or TAZ.

28. The method of claim 19, wherein the disease or condition of the control subject is selected from the group consisting of cancer, myopathy, muscular dystrophy, myotonic dystrophy, and chronic obstructive pulmonary disorder.

29-32. (canceled)
